(19) United States
(12) Patent Application Publication
(10) Pub. No.: US 2008/0254135 A1
(43) Pub. Date: Oct. 16, 2008

(54) RESVERATROL-CONTAINING COMPOSITIONS FOR GENERAL HEALTH AND VITALITY

(76) Inventors: MARVIN HEUER, MISSISSAUGA (CA); KEN CLEMENT, MISSISSAUGA (CA); CHAUDHURI SHAN, MISSISSAUGA (CA); MEGAN THOMAS, MISSISSAUGA (CA)

Correspondence Address: TORYS LLP, 79 WELLINGTON STREET WEST, SUITE 3000, BOX 270, TD CENTRE, TORONTO, ON, M5K 1N2, CA

(21) Appl. No.: 11/733,487

(22) Filed: Apr. 10, 2007

(51) Int. Cl. A61K 36/87; A61K 31/352; A61K 35/60; A61P 3/10; A61P 9/00; A61P 35/00; A61K 36/45; A61K 36/73

(52) U.S. Cl. 424/523; 424/732; 424/765; 424/766; 514/453

(57) ABSTRACT

The present invention is directed towards an orally administered composition comprising multiple plant extracts, which are characterized as having high ORAC values, and promote an increase in a mammal's dietary intake of antioxidant and polyphenols to a therapeutically effective amount. The ingredients of the present compositions represent a broad spectrum of polyphenols providing antioxidant activity for the purpose of improving general health and vitality by enhancing cardioprotection and immune system functions. The composition of the present invention comprises an extract of Polygonum cuspidatum and an extract of grape skin. In additional aspects of the present invention, one or more of Red wine extract, piceantannol, krill oil, pomegranate, mangosteen, apple, goji (wolfberry), and acai berry, cranberry, mulberry, bilberry, noni, and blueberry are added to the composition to further broaden the polyphenol spectrum. Both a composition and method are provided by the present invention.

### FIELD OF THE INVENTION

The present invention is directed towards compositions and methods for promoting an increase in a mammal's dietary intake of antioxidant and polyphenols to a therapeutically effective amount. More specifically, a composition comprising multiple extracts characterized by high ORAC values, as well as said composition supplying therapeutically effective quantities per serving of polyphenols and antioxidants is provided.

### BACKGROUND OF THE INVENTION

In recent years, the use of antioxidants to improve one's physical well-being as well as mental and physical energy has greatly increased. Improvement of general health and vitality as well as providing cardioprotective and immune system-enhancing activities are some of the many benefits which have been attributed to providing antioxidants to mammals.

Polyphenols are some of the most effective sources of antioxidants in the human diet. As such, many of the beneficial antioxidative properties of chemicals consumed in the diet of humans are attributed to polyphenols. Dietary plant-based polyphenols, such as resveratrol are reported to convey numerous health benefits including the prevention of conditions such as: diabetes, neurodegenerative disease, cardiovascular disease and cancer.

There are a vast many sources and types of polyphenols and this fact alone has contributed to the relatively slow progress on research regarding the effects of the various polyphenols (Scalbert A, Johnson I T, Saltmarsh M. Polyphenols: antioxidants and beyond. Am J Clin Nutr. January 2005;81(1 Suppl):215S-217S). Polyphenols from different sources have different structures, and thus different properties. Furthermore, similar polyphenols form different sources have different physical properties. This can affect absorption and bioavailability in the body (de Vries J H, Hollman P C, Meyboom S, Buysman M N, Zock P L, van Staveren W A, Katan M B. Plasma concentrations and urinary excretion of the antioxidant flavonols quercetin and kaempferol as biomarkers for dietary intake. Am J Clin Nutr. July 1998;68(1):60-5). These variations of polyphenols found in fruits and vegetables essentially act to provide a broad spectrum of phenolic compounds that behave similarly but have various modes of action.

Typically, supplements contain only one specific antioxidant. This approach has been show to not work as effectively as simply consuming fruits and vegetables, due to the fact that fruits and vegetables contain a range of antioxidants and polyphenols which work synergistically to confer antioxidative properties. However, the quantity of fruits and vegetables required for consumption on a daily basis in order to achieve a recommend therapeutically effective amount of antioxidants and polyphenols is substantially more than an individual does or is able to consume daily. Thus, the majority of individuals are not receiving a therapeutically effective amount of antioxidants from several polyphenols in order to meet dietary intake requirements of antioxidants.

The large scale use of antioxidants in dietary and nutritional compositions has led to the development of a tool providing a meaningful measure of the antioxidant capacity of various substances. This need led to the development of a standardized method for measuring and representing the antioxidant capacities of various foods. Oxygen Radical Absorbance Capacity (ORAC) has become the standard unit of measurement for representing antioxidant capacity of various substances. This assay is a comparison of the antioxidant capacity of the test sample relative to the antioxidant capacity of a standard antioxidant (trolox, a vitamin E analogue). Final results for test samples are published as trolox equivalents (TE), and in most cases as micromole (μmol) TE, either per gram or per 100 grams.

Typically, the normal dietary intake of a person consuming the recommended 5 serving of fruits or vegetables a day would have an ORAC value of between 1600 and 2100 μmol TE. However, according to the United States Department of Agriculture a minimum daily intake of between 3000 and 5000 ORAC units are recommended. In order to meet the recommended dietary intake (RDI) of antioxidants, it is very impractical for an individual to obtain, transport and consume enough fruits and vegetables on a daily basis to meet this requirement. A busy modern life-style has made it impossible for individuals to appropriately plan and find the time to eat the at least 10 servings, 7.5 cups of 100% fruit juice, or 10 cups of raw leafy vegetables daily to supply the recommended amount of antioxidants. Additionally, the intake of this amount of food would greatly increase an individual's daily caloric intake which is know to lead to obesity and associated maladies. Advantageously, the present invention provides a surplus of the recommended daily requirements of antioxidants from several polyphenols and other sources in a convenient nutritional supplement format.

### SUMMARY OF THE INVENTION

The present invention is directed towards an orally administered composition comprising at least a therapeutically effective amounts of plant extracts and one or more plant sources, these components having high ORAC values and supplying polyphenols and antioxidants in amounts equal to or greater than the recommended daily requirements found in fruits and vegetables. The ingredients of the present composition represent a broad spectrum of polyphenols providing antioxidant activity for the purpose of improving general health and vitality by enhancing cardioprotective and immune system functions. Both a composition and method are provided by the present invention.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards compositions and methods for promoting an increase in a mammal's dietary intake of antioxidant and polyphenols to a therapeutically effective amount, similar to that of the RDI obtained by consuming fruits and vegetables. One aspect of the present invention is directed towards promoting general health and vitality in an individual by providing cardioprotective and immune system-enhancing activities. The compositions being comprised of fruit and plant sources containing a broad spectrum of polyphenols and antioxidants. The composition in various aspects may further comprise additional ingredients to support general health; particularly ingredients with known antioxidant activity such as alpha-lipoic acid and krill oil.

In a preferred embodiment of the present invention, the composition is comprises plant sources having a broad spectrum of polyphenols and antioxidants for the purpose of promoting general health and vitality in an individual.

In an embodiment of the present invention, the composition comprises resveratrol and derivatives thereof from multiple sources in combination with other ingredients to further broaden the polyphenols spectrum. Particularly, ingredients with known antioxidant activity for the purpose of promoting general health and vitality in an individual are desired. Other said ingredients include but are not limited to Red wine extract, piceantannol, krill oil, pomegranate, mangosteen, apple, goji (wolfberry), and agai berry, cranberry, mulberry, bilberry, noni, and blueberry.

In yet another embodiment of the present invention, the composition comprises resveratrol and derivatives thereof from multiple sources in combination with one or more forms of alpha-lipoic acid and derivatives thereof. Additionally, the composition may include other ingredients supporting general health, particularly ingredients with known antioxidant activity for the purpose of promoting general health and vitality in an individual.

As used herein, the term ‘nutritional composition’ includes dietary supplements, diet supplements, nutritional supplements, supplemental dietary compositions or those similarly envisioned and termed compositions not belonging to the conventional definition of pharmaceutical interventions as is known in the art. Furthermore, ‘nutritional compositions’ as disclosed herein belong to category of compositions having at least one physiological function when administered to a mammal by conventional routes of administration.

As used herein, “high ORAC value” or other terms directed to a high ORAC value are understood to represent an ORAC value of at least about 1000 per gram for extracts. In contrast, a high ORAC value for non-extracts is typically at least about 4 per gram of fruit or vegetable. For example, fruits known to have a high ORAC value include blueberries with 24 ORAC units per gram, blackberries with 20 ORAC units per gram, plums with 9.5 ORAC units per gram, and white grapes with 4.5 ORAC units per gram. Extracts are known to have ORAC values between about 200 to about 600 times greater then that of the non-extract forms of the substances.

As used herein, a “free radical” is understood to represent an atom or molecule which bears an unpaired electron and is extremely reactive. Further it is understood that free radicals are capable in engaging in rapid chain reactions that destabilize other molecules and generate many more free radical, i.e. are self-propagating. In living tissues, free radicals are able to damage cells, accelerate the progression of cancer, cardiovascular disease, and age-related diseases. Reactive oxygen species are a common form of free radicals found in the body.

As used herein, “polyphenol” is understood to represent a group of chemical substances that are found in plants and are characterized by the presence of more then one phenol group, as known in the art, per molecule. Further it is understood that polyphenols are effective at reducing levels of free radicals in the body. This function is a result of a polyphenols' ability to both directly neutralize reactive oxidants and by direct interaction of polyphenols with receptors or enzymes that are involved in signal transduction and may result in modification of the redox status of the cell, thus triggering redox-dependent reactions.

### Resveratrol

Resveratrol is a polyphenol found in many plant sources, most notably in grape skins, grape juice and red wine. Additionally, one of the most abundant sources is from the roots of Polygonum cuspidatum. However, resveratrol is also found in other fruits such as Mulberry, Bilberry, Blueberry and Cranberry. In plants, the biological function of resveratrol is as an antibiotic to fight infection. As a component of the diet, either as a constituent of plant-based foods or as a nutritional supplement, resveratrol has been reported to confer many health benefits.

The main beneficial function of polyphenols from plant sources is generally attributed to antioxidant activity. Resveratrol has shown potent in vitro antioxidant activity (O'Brien N M, Carpenter R, O'Callaghan Y C, O'Grady M N, Kerry J P. Modulatory effects of resveratrol, citroflavan-3-ol, and plant-derived extracts on oxidative stress in U937 cells. J Med Food. 2006 Summer;9(2):187-95). The antioxidant activity has been experimentally linked to the cardioprotective effects of resveratrol. Indeed, resveratrol has been theorized to explain the “French Paradox”—the observation that some populations, despite a diet high in fat, have a low incidence of heart disease (Kopp P. Resveratrol, a phytoestrogen found in red wine. A possible explanation for the conundrum of the ‘French paradox’? European Journal of Endocrinology 138:619-620, 1998); the aforementioned populations alos consuming red wine regularly. The ability of resveratrol to inhibit low density lipoprotein (LDL) oxidation (Frémont L, Belguendouz L, Delpal S. Antioxidant activity of resveratrol and alcohol-free wine polyphenols related to LDL oxidation and polyunsaturated fatty acids. Life Sci. 1999; 64:2511-2521) may be the main factor in preventing heart disease. Resveratrol has also been shown to inhibit smooth muscle cell proliferation (Zou J, Huang Y, Chen Q, et al. Suppression of mitogenesis and regulation of cell cycle traverse by resveratrol in cultured smooth muscle cells. Int J Oncol. 1999; 15:647-651; Poussier B, Cordova A C, Becquemin J P, Sumpio B E. Resveratrol inhibits vascular smooth muscle cell proliferation and induces apoptosis. J Vasc Surg. December 2005;42(6):1190-7); which is considered necessary for arterial plaque formation a mechanism which may also prevent heart disease. Resveratrol has further been shown to induce CYP1A1 enzyme activity, which protects against disease and oxidative stress (Ciolino H P, Yeh G C. Inhibition of aryl hydrocarbon-induced cytochrome P450 1A1 enzyme activity and CYP1A1 expression by resveratrol. Mol Pharmacol. 1999; 56:760-767). The cardioprotective effects of resveratrol are thought to be a form of ‘preconditioning’, a method which is thought to be most effective in the prevention of heart disease (Das D K, Maulik N. Resveratrol in cardioprotection: a therapeutic promise of alternative medicine. Mol Interv. February 2006;6(1):36-47).

Resveratrol has also shown efficacy at modulating the immune system as manifested by anti-inflammatory, anti-allergic and anti-cancer activity (Pervaiz S. Resveratrol: from grapevines to mammalian biology. FASEB J. November 2003;17(14):1975-85). This aspect of resveratrol may involve the ability to affect specific signaling pathways such as inhibiting Nuclear Factor-kappaB (NFkB) (Manna S K, Mukhopadhyay A, Aggarwal B B. Resveratrol suppresses TNF-induced activation of nuclear transcription factors NF-kappa B, activator protein-1, and apoptosis: potential role of reactive oxygen intermediates and lipid peroxidation. J Immunol. Jun. 15, 2000;164(12):6509-19).

In addition to all the beneficial effects ascribed to resveratrol which may indirectly impact length and quality of life, resveratrol has also been shown to directly modulate members of the silent information regulator (SIR) family of proteins which contribute the regulation of life span in addition to many other important biological functions (Borra M T, Smith B C, Denu J M. Mechanism of human SIRT1 activation by resveratrol. J Biol Chem. Apr. 29, 2005;280(17):17187-95).

Natural and synthetic analogs of resveratrol have been described which offer similar or possibly enhanced effects compared to natural forms and naturally derived resveratrol. Analogues of resveratrol have been shown to effects pathways involved in cancer (Wolter F, Ulrich S, Stein J. Molecular mechanisms of the chemopreventive effects of resveratrol and its analogs in colorectal cancer: key role of polyamines? J Nutr. December 2004;134(12):3219-22). Several synthesized resveratrol analogs have been shown to have anti-inflammatory activity (Chen G, Shan W, Wu Y, Ren L, Dong J, Ji Z. Synthesis and anti-inflammatory activity of resveratrol analogs. Chem Pharm Bull (Tokyo). December 2005;53(12):1587-90).

For the purposes of the present invention, resveratrol may be derived from Polygonum Cuspidatum, an extract of Grape Skin, an extract of cranberry, an extract of mulberry, an extract of blueberry, and extract of bilberry, red wine extract, and Vitus vinifera. Additionally, the natural analog piceantannol, may be included to synergistically or additionally contribute to the effects achieved by administration of a broad spectrum of polyphenols aiding in the achievement of RDI levels of antioxidants.

Furthermore, the polyphenols, having antioxidant properties, are also contained in extracts of the aforementioned ingredients. The additional polyphenols serve to broaden the polyphenols spectrum to magnitudes similar to those found in fruits and vegetables.

It is herein understood by the inventors that resveratrol and resveratrol analogs contribute to an overall well-being and good health via at least the mechanisms outlined above.

### Proanthocyanidins

Proanthocyanidins, also known in the industry as leucoanthocyanidins, leucodelphinins, leucocyanins, anthocyanogens, epicatechin-catechin polymers or procyanins are a class of flavonoids known for their antioxidant abilities. Proanthocyanidins are polymers made from multiple anthocyanidin-like molecules known as flavanols, which under acidic conditions are broken apart to yield their constituent anthocyanidins.

For the purposes of the present invention, proanthocyanidins may be derived from an extract of Grape Skin, red wine extract, agai berry.

It is herein understood by the inventors that proanthocyanidins contribute to an overall well-being and good health via at least the mechanisms outlined above.

### Anthocyanidins

Possessing antioxidant properties, Anthocyanidins are polyphenols which are found in brightly colored fruits and vegetables. They are known to have free radical scavenging abilities. The consumption of Anthocyanidins is associated with a reduced risk of several degenerative diseases, such as for example, e.g., cancer, diabetes, and heart disease. Moreover, consumption of a diet high in antioxidants and low in fat has been shown to reduce the risk of obesity as well as insulin resistance. Similarily, the compounds of several varieties of grape skins have been shown to also possess polyphenols and antioxidant properties.

As mentioned above, Anthocyanidins are the polyphenol pigments responsible for the red, blue and purple colors of plants, including fruits and vegetables. As such, Grape skins are known to be a particularly good source of Anthocyanidins (Fuhrman B, Volkova N, Suraski A, Aviram M. White wine with red wine-like properties: increased extraction of grape skin polyphenols improves the antioxidant capacity of the derived white wine. J Agric Food Chem. July 2001;49(7):3164-8.). It has been demonstrated that Anthocyanidins possess antioxidant activity in vitro (Stintzing F C, Stintzing A S, Carle R, Frei B, Wrolstad R E. Color and antioxidant properties of cyanidin-based anthocyanin pigments. J Agric Food Chem. Oct. 9, 2002;50(21):6172-81.) as well as inhibit the proliferation of human cancer cell lines (Vareed S K, Reddy M K, Schutzki R E, Nair M G. Anthocyanidins in Cornus alternifolia, Cornus controversa, Cornus kousa and Cornus florida fruits with health benefits. Life Sci. Jan. 11, 2006;78(7):777-84.).

Ingested Anthocyanidins have been shown to be detectable in the plasma and brain as intact molecules 10 minutes after ingestion by rats (Passamonti S, Vrhovsek U, Vanzo A, Mattivi F. Fast access of some grape pigments to the brain. J Agric Food Chem. Sep. 7, 2005;53(18):7029-34.) and to be excreted in the urine of both rats and humans (McGhie T K, Ainge G D, Barnett L E, Cooney J M, Jensen D J. Anthocyanin glycosides from berry fruit are absorbed and excreted unmetabolized by both humans and rats. J Agric Food Chem. Jul. 30, 2003;51(16):4539-48.) indicating likely bioavailability. The phenols in red wine are believed to be responsible for the coronary health benefits attributed to wine and the ‘French paradox’ where the diet is relatively high in fat (Nigdikar S V, Williams N R, Griffin B A, Howard A N. Consumption of red wine polyphenols reduces the susceptibility of low-density lipoproteins to oxidation in vivo. Am J Clin Nutr. August 1998;68(2):258-65.). However, the French show a low incidence of coronary disease as compared to what would normally be expected with such a diet.

Anthocyanidins in accordance with the present invention may be derivatived or obtained from various sources such as for example, but not limited by, e.g., Grape Skin, an extract of cranberry, an extract of mulberry, an extract of blueberry, and extract of bilberry, red wine extract, apple, and Vitus vinifera Furthermore, Anthocyanidins or derivatives may be obtained from any red, blue or purple fruit, vegetable, or flower.

### Blueberry Extract

Blueberries are a group of flowering plants in the genus Vaccinium. The species are native to North America and eastern Asia and contain a high amount of anthocyanins, potent antioxidants. Anthocyanins are water-soluble flavonoid pigments that appear red to blue depending on pH. Flavonoids have been reported to demonstrate their benefits in lowering oxidative stress and beneficial effects on cardiovascular and chronic inflammatory diseases.

### Cranberry Extract

Cranberries are a group of evergreen dwarf scrubs found in acidic bogs throughout the cooler parts of the Northern Hemisphere. Cranberries are a major commercial crop in the United States and Canada where they are processed into consumer products, like juices and sauces. Cranberries are a source of flavonoids, chemicals which are known to provide certain health benefits to the immune system. More specifically cranberries are a source of proanthocyanidins, which are polymers made from multiple anthocyanidin-like molecules known as flavanols. These are called proanthocyanidins because under acidic conditions they are broken apart to yield their constituent anthocyanidins.

### Pomegranate

Pomegranate (Punica granatum) fruit is a source of vitamin C, folic acid and antioxidant polyphenols. The bioavailability and antioxidant activity of pomegranate extract has been shown in human studies (Mertens-Talcott S U, Jilma-Stohlawetz P, Rios J, Hingorani L, Derendorf H. Absorption, metabolism, and antioxidant effects of pomegranate (Punica granatum I.) polyphenols after ingestion of a standardized extract in healthy human volunteers. J Agric Food Chem. Nov. 15, 2006;54(23):8956-61 Abstract).

In mouse and rat models, pomegranate extract has been shown to improve lipid metabolism and improve blood lipid parameters by lowering blood lipid levels (Huang T H, Peng G, Kota B P, Li G Q, Yamahara J, Roufogalis B D, Li Y. Pomegranate flower improves cardiac lipid metabolism in a diabetic rat model: role of lowering circulating lipids. Br J Pharmacol. July 2005;145(6):767-74) and by reducing the accumulation of triglycerides in the liver (Arao K, Wang Y M, Inoue N, Hirata J, Cha J Y, Nagao K, Yanagita T. Dietary effect of pomegranate seed oil rich in 9cis, 11trans, 13cis conjugated linolenic acid on lipid metabolism in obese, hyperlipidemic OLETF rats. Lipids Health Dis. Dec. 9, 2004;3:24). In diabetic humans with hyperlipidemia, concentrated pomegranate juice reduced total cholesterol and LDL levels (Esmaillzadeh A, Tahbaz F, Gaieni I, Alavi-Majd H, Azadbakht L. Concentrated pomegranate juice improves lipid profiles in diabetic patients with hyperlipidemia. J Med Food. 2004 Fall;7(3):305-8).

It is herein understood by the inventors that pomegranate contributes to an overall well-being and good health via at least the mechanisms outlined above.

### Mangosteen

Mangosteen (Garcinia mangostana) is a tropical tree the fruit of which has been explored for health and therapeutic benefits.

Mangosteen extracts have been shown to have antioxidant activity (Jung H A, Su B N, Keller W J, Mehta R G, Kinghorn A D. Antioxidant xanthones from the pericarp of Garcinia mangostana (Mangosteen). J Agric Food Chem. Mar. 22, 2006;54(6):2077-82 Abstract). This antioxidant activity may be responsible for neuroprotective activity (Weecharangsan W, Opanasopit P, Sukma M, Ngawhirunpat T, Sotanaphun U, Siripong P. Antioxidative and neuroprotective activities of extracts from the fruit hull of mangosteen (Garcinia mangostana Linn.). Med Princ Pract. 2006;15(4):281-7 Abstract). Furthermore, specific constituents of Mangosteen have demonstrated antibiotic activity (Suksamrarn S, Suwannapoch N, Phakhodee W, Thanuhiranlert J, Ratananukul P, Chimnoi N, Suksamrarn A. Antimycobacterial activity of prenylated xanthones from the fruits of Garcinia mangostana. Chem Pharm Bull (Tokyo). July 2003;51(7):857-9) which may involve enhancement of immune function or serve to lighten immune function load during times of challenge. Additionally, Mangosteen is capable of inhibiting the release histamine (Nakatani K, Atsumi M, Arakawa T, Oosawa K, Shimura S, Nakahata N, Ohizumi Y. Inhibitions of histamine release and prostaglandin E2 synthesis by mangosteen, a Thai medicinal plant. Biol Pharm Bull. September 2002;25(9):1137-41), which is involved in immune reactions, particularly immune inflammation.

It is herein understood by the inventors that mangosteen contributes to an overall well-being and good health via at least the mechanisms outlined above.

### Krill Oil

Krill oil, similar to fish oil, contains both of the omega-3 fats eicosapentanoic acid (EPA) and docosahexanoic acid (DHA). In fish oil these omega-3 fatty acids are found in the triglyceride form whereas in krill oil they are hooked up in a double chain phospholipid structure. Each part of the phospholipid is punctuated by a molecule of astaxanthin, an extremely potent antioxidant. Since the active ingredients of krill oil have a phospholipid structure they are much more absorbable and can easily enter the mitochondria and the cellular nucleus.

Krill oil also contains a number of potent antioxidants, such as vitamin E, vitamin A, and vitamin D. The antioxidant potency of krill oil in terms of ORAC values was found to be about 378 μmol/g, 47 times more potent than fish oil, which typically has an ORAC value of about 8 μmol/g. Since krill oil is such a potent antioxidant, its inclusion in supplements for well-being and good health would synergistically or additionally contribute to the effects achieved by administration of a broad spectrum of polyphenols aiding in the achievement of RDI levels of antioxidants.

In a randomized, double blind, placebo-controlled study completed on ninety patients diagnosed with cardiovascular disease (Deutsch L. Evaluation of the effect of neptune krill oil on chronic inflammation and arthritic symptoms. J Am Coll Nutr. February 2007;26(1):39-48 (Abstract)), it was shown that a daily dose of 300 mg of krill oil, over a period of 7 to 14 days, significantly inhibits inflammation.

It is herein understood by the inventors that due to krill oil's potent antioxidant properties, it will contribute to decreasing inflammation.

### Goji

Wolfberries the common name for Lycium barbarum, also called Goji berries are a highly nutritionally rich fruit originally grown in Europe and now cultivated in China. They contain all 8 essential amino acids, up to 21 trace minerals and a variety of vitamins, and have been used in traditional Chinese medicine for nearly 2,000 years. The active polysaccharide extracts are believed to enhance immune system function, improve eyesight, protect the liver, boost sperm production, and improve circulation.

Wolfberry polysaccharides treatment of age-related oxidative stress in mice has been shown to improve the total antioxidant capacity as well as improved immune function (Li X M, Ma Y L, Liu X J. Effect of the Lycium barbarum polysaccharides on age-related oxidative stress in aged mice. J Ethnopharmacol. Dec. 28, 2006; [Epub ahead of print] (Abstract)). The same study also illustrated that the antioxidant activity of the wolfberries could be increased with co-treatment of vitamin C.

Administration of Lycium barbarum polysaccharides increased the expression of interleukin-2 and tumor necrosis factor-alpha at both mRNA and protein levels in a dose-dependent manner (Gan L, Zhang S H, Liu Q, Xu H B. A polysaccharide-protein complex from Lycium barbarum upregulates cytokine expression in human peripheral blood mononuclear cells. Eur J Pharmacol. Jun. 27, 2003;471(3):217-22(Abstract)). The results suggest that Lycium barbarum polysaccharides may induce immune responses and possess potential therapeutic efficacy in cancer.

It is herein understood by the inventors that goji (wolfberry) contributes to an overall well-being and good health via at least the mechanisms outlined above.

### Alpha-lipoic Acid

Alpha lipoic acid is potent antioxidant that has been studied as a therapeutic agent for a number of diseases including diabetes, atherosclerosis and neurodegenerative conditions (Bilska A, Wlodek L. Lipoic acid—the drug of the future? Pharmacol Rep. October-September 2005;57(5):570-7). Alpha-lipoic acid is efficacious as an antioxidant for two important reasons. First, alpha-lipoic acid, while possessing its own antioxidant activity, works synergistically with the antioxidant activity of vitamin C and vitamin E. Second, alpha-lipoic acid is unusual among anti-oxidants in that it is both water and fat soluble, greatly adding to its antioxidation potential.

Alpha-lipoic acid, although shown to have beneficial effects when administered does have drawbacks related to consumption. A preferred route of administration for alpha-lipoic acid is oral; however it has long been known that severe irritation of the upper potion of the esophagus, namely the throat occurs via this administration route. A method of production of the trometamol salt of alpha lipoic acid, is disclosed in U.S. Pat. No. 3,562,273 incorporated herein in it's entirely by reference. The patent discloses that the trometamol salt of alpha lipoic acid is very soluble in water and has a pH of 6.5 in a 0.1M aqueous solution. Similarly, additional salts of alpha-lipoic acid may be used to improve the efficacy of the composition.

For the purposes of the present invention, forms of alpha lipoic acid may be selected from potassium alpha-lipoic acid, sodium alpha-lipoic acid, trometamol alpha-lipoic acid, alpha-lipoic acid nicotinamide, dihydrolipoic acid, and Thioctamide.

It is herein understood that, administration of a composition comprising multiple plant extracts, characterized by high ORAC values, supplying therapeutically effective quantities, per serving, of polyphenols will result in the protection and support of cardioprotective and immune system functions, thus promoting general health and vitality of a mammal. The composition and format of the present invention will supply at least the RDI of antioxidants according to the ORAC scale to user similar to that of consuming the RDI of fruits and vegetables.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage forms of the present invention are as a caplet or as a liquid capsule.

U.S. application Ser. No. 11/709,526 entitled “Method for Increasing the Rate and Consistency of Bioavailability of Supplemental Dietary Ingredients”, filed Feb. 21, 2007, which is herein fully incorporated by reference discloses a method of improving the rate of bioavailability of compounds by increasing the rate of solubility. The increased rate of bioavailability of a compound or ingredients is achieved via a reduction in particle size using a “fine-milling” technique. Any acceptable fine-milling technique will result in the fine-milled particles having an average particle size of between about 50 microns to about 2 microns. The reduction in size of the particle increases the surface area-to-volume ratio of each particle, thus increasing the rate of dissolution, thereby improving the rate of absorption.

As used herein, the terms “fine-milled” and/or “fine-milling” refer to the process of micronization. Micronization is a mechanical process which involves the application of force to a particle, thereby resulting in a reduction in the size of said particle.

As used herein, the term “particle size” refers to the diameter of the particle. The term “average particle size” means that at least 50% of the particles in a sample will have the specified particle size. Preferably, at least 80% of the particles in a given sample will have the specified particle size, and more preferably, at least 90% of the particles in a given sample will have the specified particle size.

According to various embodiments of the present invention, the nutritional supplement may be administered in a dosage form with controlled release characteristics i.e. time-release. Furthermore, the controlled release may be in the form of either delayed release of active constituents or gradual, prolonged release. The goal of such strategies is to extend the period of bioavailability or to meet a specific window for optimal bioavailability. Advantageously the nutritional supplement may be administered in the form of a multi-compartment capsule which combines both immediate release and time-release characteristics. Individual components of the nutritional supplement may be contained in differential compartments of such a capsule such that specific components are released rapidly while others are time-released. Alternatively, a uniform mix of the various components of the present invention may be divided into both immediate release and time-release compartments to provide a multi-phasic release profile.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

Additionally, by way of ingestion of the nutritional composition of the present invention, a method providing the body of a user with at least the RDI of antioxidants is achieved. The method of the present invention comprises at least the step of administering to an individual a therapeutically acceptable amount of the composition of the present invention.

The present nutritional composition or those similarly envisioned by one of skill in the art may be utilized in methods to enhance the general health and vitality of an individual by protecting and supporting cardioprotective and immune system function through antioxidant activity provided by specifically chosen ingredients.

Although the following examples illustrate the practice of the present invention in several of its embodiments, the examples should not be construed as limiting the scope of the invention. Other embodiments will be apparent to one of skill in the art from consideration of the specifications and example.

### EXAMPLE 1

A nutritional supplement in the form of a caplet to be taken at least once daily, to provide antioxidant polyphenols conveniently and in excess of RDI. One serving of the nutritional supplement contains the following ingredients:

About 0.24 g of Polygonum cuspidatum standardized for 50% resveratrol, about 0.001 g of Polygonum cuspidatum standardized for 98% resveratrol, about 0.001 g of grape skin extract standardized to proanthocyanidins, and about 0.01 g of red wine extract standardized for 25% proanthocyanidins.

### EXAMPLE 2

A nutritional supplement in the form of a caplet to be taken at least once daily, to provide antioxidant polyphenols conveniently and in excess of RDI. One serving of the nutritional supplement contains the following ingredients:

About 0.24 g of Polygonum cuspidatum standardized for 50% resveratrol, about 0.001 g of Polygonum cuspidatum standardized for 98% resveratrol, about 0.001 g of grape skin extract standardized to proanthocyanidins, about 0.001 g of piceatannol (Rheum udulatum), about 0.01 g of red wine extract standardized for 25% proanthocyanidins, and about 0.01 g of Neptune Krill oil.

### EXAMPLE 3

A nutritional supplement in the form of a caplet to be taken at least once daily, to provide antioxidant polyphenols conveniently and in excess of RDI. One serving of the nutritional supplement contains the following ingredients:

About 0.3 g of Polygonum cuspidatum standardized for 50% resveratrol, about 0.001 g of Polygonum cuspidatum standardized for 98% resveratrol, about 0.05 g of grape skin extract standardized for proanthocyanidins, about 0.001 g of cranberry extract standardized for resveratrol, about 0.001 g of mulberry extract standardized for resveratrol, about 0.001 g of blueberry extract standardized for resveratrol, about 0.001 g of bilberry extract standardized for resveratrol, about 0.001 g of red wine extract standardized for 25% proanthocyanidins, about 0.001 g of Acai berry, about 0.001 g of Gogi (Wolfberry), about 0.001 g of apple extract, about 0.001 g of pomegranate extract, about 0.001 g of mangosteen extract, and about 0.001 g of alpha-lipoic acid.

### Extensions and Alternatives

In the foregoing specification, the invention has been described with a specific embodiment thereof, however, it will be evident that various modifications and changes may be made thereto without departing from the broader spirit and scope of the invention.

What is claimed:

1. An oral composition comprising a therapeutically effective amount of an extract of Polygonum cuspidatum and an extract of grape skin wherein the extract of Polygonum cuspidatum and the extract of grape skin are characterized by having a therapeutically effective amount of polyphenols and antioxidants per serving.

2. The composition of claim 1, wherein the extract of Polygonum cuspidatum and the extract of grape skin are further characterized by having high ORAC values.

3. The composition of claim 1, further comprising one or more ingredients selected from the group consisting of: red wine extract, piceantannol, krill oil, pomegranate, mangosteen, apple, goji (wolfberry), and acai berry, cranberry, mulberry, bilberry, noni, and blueberry.

4. The composition of claim 3 wherein the one or more ingredients afford a range of antioxidants and polyphenols acting synergistically to provide a mammal with at least the minimum recommended daily ORAC unit intake.

5. The composition of claim 1, further comprising alpha-lipoic acid and derivatives thereof.

6. The composition of claim 5 wherein the alpha-lipoic acid derivatives are selected from the group consisting of potassium alpha-lipoic acid, sodium alpha-lipoic acid, trometamol alpha-lipoic acid, alpha-lipoic acid nicotinamide, dihydrolipoic acid, and Thioctamide.

7. The composition of claim 1 further comprising at least 100 mg of polyphenols per serving.

8. A method of providing a mammal with at least the minimum recommended daily ORAC units; the method comprising the step of administering to the mammal a composition comprising a therapeutically effective amount of an extract of Polygonum cuspidatum and an extract of grape skin wherein the extract of Polygonum cuspidatum and the extract of grape skin are characterized by having a therapeutically effective amount of polyphenols and antioxidants per serving.

9. The method of claim 8, wherein the extract of Polygonum cuspidatum and the extract of grape skin are further characterized by having high ORAC values.

10. The method of claim 8, further comprising one or more ingredients selected from the group consisting of: red wine extract, piceantannol, krill oil, pomegranate, mangosteen, apple, goji (wolfberry), and acai berry, cranberry, mulberry, bilberry, noni, and blueberry.

11. The method of claim 10 wherein the one or more ingredients afford a range of antioxidants and polyphenols acting synergistically to provide a mammal with at least the minimum recommended daily ORAC unit intake.

12. The method of claim 8, further comprising alpha-lipoic acid and derivatives thereof.

13. The method claims 12 where the alpha-lipoic acid derivative are selected from the group consisting of potassium alpha-lipoic acid, sodium alpha-lipoic acid, trometamol alpha-lipoic acid, alpha-lipoic acid nicotinamide, dihydrolipoic acid, and Thioctamide.

14. The method of claim 8 further comprising at least 100 mg of polyphenols per serving.

15. An oral composition comprising at least 0.24 g of an extract of Polygonum cuspidatum standardized to 50% resveratrol, at least 0.001 g of an extract of Polygonum cuspidatum standardized to 98% resveratrol and at least 0.001 g of an extract of grape skin standardized to 25% proanthocyadins

16. The composition of claim 15 wherein the extract of Polygonum cuspidatum and the extract of grape skin are characterized by having a therapeutically effective amount of polyphenols and antioxidants per serving.

17. The composition of claim 15, wherein the extract of Polygonum cuspidatum and the extract of grape skin are further characterized by having high ORAC values.
